(19) United States
(12) Patent Application Publication
(10) Pub. No.: US 2026/0137559 A1
(43) Pub. Date: May 21, 2026

(54) INTRAOCULAR IMPLANTS AND METHODS FOR IMPLANTING INTRAOCULAR IMPLANTS

(71) Applicant: AMO GRONINGEN B.V., Groningen (NL)

(72) Inventors: Theophilus Bogaert, Groningen (NL); Marrie van der Mooren, Engelbert (NL)

(21) Appl. No.: 18/955,722

(22) Filed: Nov. 21, 2024

(51) Int. Cl. A61F 9/007

(52) U.S. Cl. CPC A61F 9/00781; A61F 2210/0014; A61F 2220/0008; A61F 2230/0039

(57) ABSTRACT

An intraocular implant for implantation in an iris of the eye is disclosed. The intraocular implant may include a tubular member at least partially formed of a shape memory material to enable the tubular member to transition between a first shape and a second shape. The tubular member may have a first retaining element, a second retaining element, a shaft portion extending between the first and second retaining portions, and at least one lumen extending therethrough. The shaft portion of the tubular member may have a length substantially equal to or less than a thickness of the iris. The shaft may have a smaller cross-section than the first and second retaining elements when the tubular member is in the second state.

### FIELD OF INVENTION

The present disclosure is directed to the field of medical devices, and in particular, intraocular implants for the treatment of glaucoma, and methods for implanting intraocular implants in an eye.

### BACKGROUND

This background description is provided for the purpose of generally presenting the context of the disclosure. Unless otherwise indicated herein, material described in this section is neither expressly nor impliedly admitted to be prior art to the present disclosure or the appended claims.

Intraocular pressure in the eye is maintained by the formation and drainage of aqueous humor. The aqueous humor is a clear, colorless fluid that fills the anterior and posterior chambers of the eye. The aqueous humor is formed by the ciliary body in the eye and is a carrier of nutrients for the lens. In addition, the aqueous humor provides a continuous stream into which surrounding tissues can discharge the waste products of metabolism.

The aqueous humor produced in the ciliary process circulates from the posterior chamber to the anterior chamber of the eye through the pupil and is absorbed through the trabecular meshwork, a plurality of criss-crossing collagen cords covered by endothelium. Once through the trabecular meshwork, the aqueous humor passes through Schlemm's canal and into venous circulation. The rate of aqueous humor outflow through the trabecular meshwork in a normal eye is typically 2.1 μL/min. Intraocular pressure in the eye is maintained by the formation and drainage of the aqueous humor. All the tissues within the corneoscleral coat covering the eyeball are subject to this pressure, which is higher than pressure exerted on tissues at other locations in the body.

Glaucoma is a progressive neurodegenerative disease of the eye mostly caused by a gradual increase of intraocular pressure and characterized by a gradual loss of peripheral vision. This increase in pressure is most commonly caused by stenosis or blockage of the aqueous humor outflow channel, resulting in excessive buildup of aqueous humor in the eyeball. Other causes include an increase in venous pressure outside the eye which is reflected back through the drainage channels and an increase production of aqueous humor. In a “normal” eye, intraocular pressure ranges from 8 to 21 mmHg. In an eye with glaucoma, intraocular pressure can range between normal pressures up to as much as 50 mmHg. This increase in intraocular pressure produces gradual and permanent loss of vision in the afflicted eye.

Existing corrective methods for the treatment of glaucoma include medications or drugs, surgery, and implants. For example, miotic drugs lower intraocular pressure by facilitating aqueous outflow. Beta blockers, epinephrine products, and carbonic anhydrase inhibitors, which inhibit production of the aqueous, are also commonly used in pharmacological glaucoma treatment. Steroids have been used in long-term glaucoma treatment as well. However, pharmacological treatment may be prohibitively expensive to a large majority of glaucoma patients. In addition, the medications used in the treatment, in particular steroids, often have undesirable side effects including, for example, burning sensations in the eye, headaches, cardiac fluctuations and blurred vision, and many of the long-term effects resulting from prolonged use are not yet well known.

Surgical procedures have been developed in an effort to treat people with glaucoma. For example, an iridectomy, the removal of a portion of the iris, is often used in angle-closure glaucoma when there is an occlusion of the trabecular meshwork by iris contact. The opening from the removal of a piece of the iris then gives the aqueous humor free passage from the posterior chamber to the anterior chamber in the eye and eliminates the bulging of the iris that causes the angle closure. While iridectomy procedures are often successful, the tissue of the eye, however, can scar over the removed portion of the iris and the eye may revert to the pre-operative condition, thereby necessitating the need for further treatment. Further, some patients may experience undesirable visual effects (e.g., dysphotopsia), such as glare or halos, as a result of the openings in the iris. For example, halos may arise when light travels through the openings in the iris to the retina, causing increased brightness in the focal image.

For at least these reasons, it would be advantageous to develop an intraocular implant that can easily implanted into incisions or openings formed through the iris of an eye to reduce or mitigate dysphotopsia, such as halos and glare.

### SUMMARY

The present disclosure is directed to embodiments relating to medical devices, such as intraocular implants, for the treatment of eye diseases, such as glaucoma. The embodiments can be easily implanted into incisions or openings made through the iris of an eye by an iridectomy procedure. The embodiments can reduce or mitigate dysphotopsia, such as halos and glare, and other aberrant optical effects as a result of the openings in the iris of the eye. For example, the embodiments may reduce the amount or intensity of light that travels through the openings in the iris to the retina of the eye. Further, the embodiments may provide a fluid pathway for the flow or drainage of aqueous humor between the anterior chamber and the posterior chamber of the eye.

The embodiments may be made of a flexible material capable of shape memory. Shape memory refers to the capability of a material to regain or recall its previous shape after subjecting to a deformation due to mechanical force or by heating and/or cooling. For example, the material may include nitinol, polymers (e.g., a polymer material with a glass transition temperature between 15-25C which will revert to its original shape after passing through its glass transition temperature), biomaterials, hydrogels, foams, rubbers, or any suitable material that may be compressed and, upon release, may enlarge and/or expand axially and/or radially to assume an initial, predetermined or preformed shape. For example, the material may be compressed or deformed in a dehydrated state and may return to its original state upon hydration. Because of the pliable construction and shape, the embodiments can be quickly and efficiently implanted in the openings in the iris of an eye without the use of sutures. As a result, the time required for implanting the embodiments in an eye may be substantially shortened or reduced. Further, the embodiments may be implanted in the eye using a delivery or injection system that uses a minimally invasive procedure.

In one aspect, an intraocular implant for implantation in an iris of the eye is disclosed. The intraocular implant may include a tubular member at least partially formed of a shape memory material to enable the tubular member to transition between a first shape and a second shape. The tubular member may have a first retaining element, a second retaining element, a shaft portion extending between the first and second retaining elements, and at least one lumen extending therethrough. The shaft portion of the tubular member may have a length substantially equal to or less than a thickness of the iris. The shaft portion may have a smaller cross-section than the first and second retaining elements when the tubular member is in the second state.

In another aspect, a method of implanting an intraocular implant into an iris of the eye is disclosed. The method may include forming an incision in the eye and placing an intraocular implant into a lumen of a sleeve of a surgical instrument. The sleeve of the surgical instrument may be configured to hold and maintain the intraocular implant in a first shape for implantation in the eye and the intraocular implant may be constructed from an expandable material. The method may also include inserting a portion of the sleeve of the surgical instrument into the eye through the incision and placing a distal end of the sleeve of the surgical instrument adjacent to an opening in the iris. Further, the method may include ejecting the intraocular implant from the lumen of the surgical instrument into the opening of the iris. The intraocular implant may be configured to expand from the first shape to a second shape when intraocular implant is ejected from the lumen. Additionally, the method may include removing the portion of the sleeve of the surgical instrument from the eye.

The foregoing summary is illustrative only and is not intended to be in any way limiting. In addition to the illustrative aspects, embodiments, and features described above, further aspects, embodiments, and features will become apparent by reference to the figures and the following detailed description.

### BRIEF DESCRIPTION OF THE DRAWINGS

FIG. 1 is a cross-sectional view of a human eye;

FIG. 2 shows an intraocular implant implanted in the eye of FIG. 1;

FIG. 3 is a perspective view of an intraocular implant for implantation in an eye, in accordance with an exemplary embodiment;

FIG. 4 is a cross-sectional view of the intraocular implant of FIG. 3;

FIG. 5 is a cross-sectional view of another an intraocular implant for implantation in an eye, in accordance with an exemplary embodiment;

FIG. 6 is a cross-sectional view of another an intraocular implant for implantation in an eye, in accordance with an exemplary embodiment;

FIG. 7 is a cross-sectional view of another an intraocular implant for implantation in an eye, in accordance with an exemplary embodiment;

FIG. 8 is a cross-sectional view of another an intraocular implant for implantation in an eye, in accordance with an exemplary embodiment;

FIG. 9 is a partial cross-sectional view of a surgical instrument for delivering and/or inserting an intraocular implant in an iris of an eye;

FIG. 10 is a partial cross-sectional view of a surgical instrument of FIG. 9 showing an intraocular implant according to one aspect of the invention partially deployed from the surgical instrument; and

FIG. 11 is a partial cross-sectional view of a surgical instrument of FIG. 9 showing the intraocular implant according to one aspect of the invention deployed from the surgical instrument.

### DETAILED DESCRIPTION

Before explaining the embodiments in detail, it should be noted that the present disclosure is not limited in its application or use to the details of construction and arrangement of parts illustrated in the accompanying drawings and description, because the illustrative embodiments may be implemented or incorporated in other embodiments, variations and modifications, and may be practiced or carried out in various ways. Furthermore, unless otherwise indicated, the terms and expressions employed herein have been chosen for the purpose of describing the embodiments of the present disclosure for the convenience of the reader and are not for the purpose of limitation.

The present disclosure is directed to intraocular implants for the treatment of eye diseases, such as glaucoma. The intraocular implants can be easily implanted into incisions or openings (e.g., holes) made through the iris of an eye by an iridectomy procedure. The incisions or openings in the iris may be made by a laser, a blade, a punch, or any other suitable device or instrument prior to implanting an intraocular implant. For example, an iridotomy procedure may use a laser to make a hole in the iris. In some embodiments, a surgical instrument may be used to create an opening or hole in the iris while implanting an intraocular implant in the iris.

The intraocular implants can reduce or mitigate dysphotopsia, such as halos and glare, and other aberrant optical effects as a result of the openings in the iris of the eye. For example, the intraocular implants may reduce the amount or intensity of light that travels through the openings in the iris to the retina of the eye. Further, the intraocular implants may provide a fluid pathway for the flow or drainage of aqueous humor between the anterior chamber and the posterior chamber of the eye.

The intraocular implants may be made of a flexible material capable of shape memory. For example, the flexible material may be compressed and, upon release, may enlarge and/or expand axially and/or radially to assume a particular shape. Because of the pliable construction and shape, the intraocular implants can be quickly and efficiently implanted in the openings in the iris of an eye without the use of sutures. As a result, the time required for implanting the intraocular implants in an eye may be substantially shortened or reduced. Further, the intraocular implants may be implanted in the eye using a delivery or injection system that uses a minimally invasive procedure.

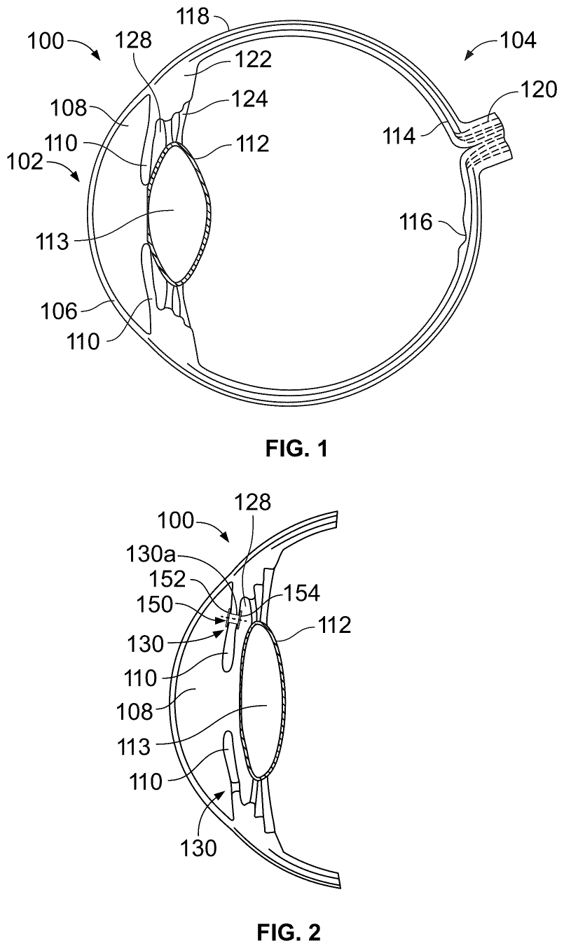

Referring now to drawings, FIG. 1 illustrates a cross-sectional view of a human eye 100. The eye 100 includes a frontal portion 102 and a rearward portion 104. The frontal portion 102 of the eye 100 is covered by a cornea 106 which encloses and forms an anterior chamber 108. The anterior chamber 108 contains aqueous fluid and is bounded at the rear by an iris 110. The iris 110 has a hole in the center, called “the pupil”, that increases and decreases in size to control the amount of light entering into the inner portions of the eye 100. The eye 100 includes a capsule or capsular bag 112 which ordinarily contains a natural crystalline lens 113. When the eye 100 focuses, the crystalline lens 113 changes shape to appropriately distribute and/or focus the light admitted through the cornea 106 and the pupil of the iris 110 to a retina 114 at the rearward portion 104 of the eye 100.

The retina 114 is composed of rods and cones which act as light receptors. The retina 114 includes a fovea 116 which is a rod-less portion that provides for acute vision. The outside of the rearward portion 104 of the eye 100 is known as the sclera 118 which joins into and forms a portion of the covering for an optic nerve 120. Images received by the retina 114 are transmitted through the optic nerve 120 to the brain. The area between the retina 114 and the capsular bag 112 is occupied by vitreous fluid. The eye 100 also includes a ciliary muscle or body 122 having zonular fibers 124 (also referred to as zonules) which are attached to the capsular bag 112.

Ocular adjustments for sharp focusing of objects viewed at different distances is accomplished by the action of the ciliary body 122 on the capsular bag 112 and the crystalline lens 113 through the zonular fibers 124. The ciliary body 122 contracts, allowing the capsular bag 112 to return to a more spherical shape for viewing objects that are nearer or closer to the viewer. When the ciliary body 122 retracts and pulls on the zonular fibers 124 to make the capsular bag 112 more discoid, objects at a distance can be viewed in proper focus.

The ciliary body 122 of the eye 100 also continuously forms aqueous humor in a posterior chamber 128 by secretion from the blood vessels. The aqueous humor flows between the posterior surface of the iris 110 and the anterior surface of the crystalline lens 113 (e.g., the capsular bag 112) through the pupil into the anterior chamber 108 and exits the eye through the trabecular meshwork, a sieve-like structure situated at the corner of the iris 110 and the wall of the eye (the corner is known as the iridocorneal angle). The aqueous humor may filter through the trabecular meshwork into Schlemm's canal, a small channel that drains into the ocular veins. A portion of the aqueous humor may rejoin the venous circulation after passing through the ciliary body 122 and eventually through the sclera 118 (the uveoscleral route). As the aqueous humor is leaving the eye, aqueous humor may flow into the eye to create a balance between the inflow of the aqueous humor and out flow of aqueous humor leaving the eye.

FIG. 2 shows an intraocular implant 150 implanted in the iris 110 of the eye 100 of FIG. 1. Prior to implantation of the intraocular implant 150, an iridectomy procedure may be performed to remove one or more portions of the iris 110 to create small incisions or openings 130 (two being shown) in the iris 110. The openings 130 may be made through the outer periphery of the iris 110. The openings 130 in the iris 110 improve the fluid flow of the aqueous humor from the posterior chamber 128 to the anterior chamber 108. The openings 130 extend through the iris 110 generally parallel to the optical axis of the eye. In some embodiments, the openings 130 may extend at angles that are non-parallel to the optical axis of the eye.

As shown in FIG. 2, the intraocular implant 150 is disposed in the opening 130a in the iris 110 created by an iridectomy procedure. The intraocular implant 150 is positioned in the opening 130a of the iris 110 such that a proximal end 152 of the intraocular implant 150 is located in the anterior chamber 108 and a distal end 154 is located in the posterior chamber 128. As such, the intraocular implant 150 can serve as a passageway for the flow of the aqueous humor through the intraocular implant 150 between the anterior chamber 108 and the posterior chamber 128. The intraocular implant 150 may include retaining elements or collars, as further described below, to anchor and retain the intraocular implant 150 through the opening 130a of the iris 110 and to prevent the intraocular implant 150 from slipping out of the opening 130a once implanted or in place.

The intraocular implant of the present invention is configured to be transitionable between a first shape or state of a reduced size and a second shape or state of an expanded size and vice-versa. For example, the first shape of the tubular member may comprise a compressed shape, a reduced shape, a compact shape, a deformed shape, a pre-deployed shape, or a dehydrated shape, and the second shape of the tubular member may comprise an uncompressed shape, an expanded shape, an initial shape, an enlarged shape, a deployed shape, or a hydrated shape. The intraocular implant can be in a first shape when the implant has a reduced or compact size (e.g., reduced radial size) in order to facilitate implantation of the intraocular implant in the eye. For example, the intraocular implant can be positioned within a lumen of a surgical instrument that can implant the intraocular implant into the eye.

The lumen of the surgical instrument may hold and maintain the implant in the first shape of reduced size until implantation. When implanting the intraocular implant into the eye, the intraocular implant may be ejected or deployed from the lumen of the surgical instrument to permit the intraocular implant to expand in size. The transition in shape of the intraocular implant can begin to change once the implant is injected or deployed into an opening in the iris of the eye as further described below. In some embodiments, the intraocular implant can also change cross-sectional shape along its length.

The transition of the intraocular implant between the first and second shapes can be implemented in various manners. The intraocular implant may be fabricated from a material capable of shape memory. For example, the material may include nitinol, shape memory polymers (e.g., a polymer material with a glass transition temperature between 15-25C which will revert to its original shape after passing through its glass transition temperature), biomaterials, hydrogels, foams, rubbers, or any suitable material that may be compressed and, upon release, enlarge and/or expand axially and/or radially to assume an initial or preformed shape. For example, the material may be compressed or deformed in a dehydrated state and may return to its original state upon hydration.

The material may expand in absence of an external force applied to the intraocular implant. For example, the material may expand in response to a release of a constraining element, such as a lumen of a surgical instrument that constrains the intraocular implant prior to deployment in an eye. In some embodiments, a portion of the intraocular implant 350 may made from expandable material while another portion of the implant may be made from rigid material. For example, a central or shaft portion of the intraocular implant may be constructed from a non-expanded material, such as a plastic, a polymer material, or any other suitable material. In some embodiments, the central part may be resilient and flexible to accommodate different iris depths or to absorb the relative movement of the posterior surface of the iris relative to its anterior (stationary) surface when the pupil is contracting. The intraocular implant may be made of a hydrogel (e.g., poly 2-hydroxyethyl methacrylate (pHEMA), collagen, polyvinyl alcohol hydrogels (PVA)), or any other suitable material. As such, when the intraocular implant is made from a hydrogel, the intraocular implant may be molded and dried in a reduced size and may expand to original size after fluid or water uptake.

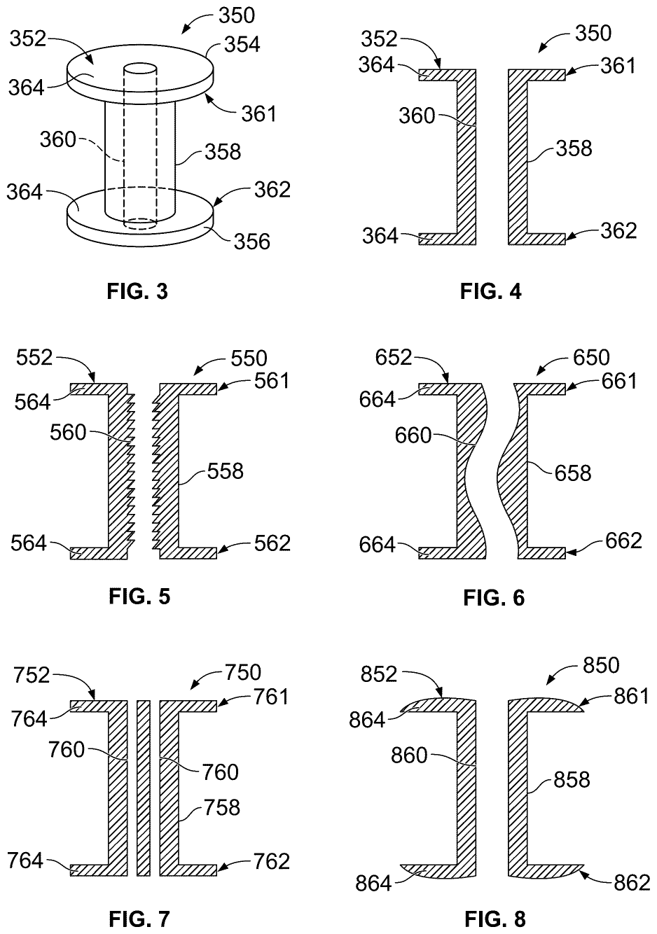

FIG. 3 illustrates a perspective view of an intraocular implant 350, in accordance with an exemplary embodiment. The intraocular implant 350 is configured to be implanted into an eye of a patient. As shown in FIG. 3, the intraocular implant 350 includes a tubular member or elongated body 352 having a first or proximal end 354, a second or distal end 356, a shaft or central portion 358, and one or more lumens or conduits extending therethrough 360. As illustrated, the tubular member 352 includes a lumen 360 that provides a pathway for fluid (e.g., aqueous humor) to flow through the tubular member 352. For example, when the tubular member 352 is positioned in an iris of the eye, the lumen 360 can serve as a passageway that enables aqueous humor to flow through the lumen 360 between an anterior chamber and a posterior chamber of an eye.

As shown in FIG. 4, the lumen 360 of the tubular member 352 has a substantially circular cross-section that is axially positioned along a central axis of the tubular member 352. In some embodiments, the cross-sectional shape of the lumen 360 may be oval, rectangular, or any other suitable shape. The outer wall of the lumen 360 may be substantially smooth or uniform. In some embodiments, the outer wall of the lumen 360 may be rough or uneven and/or may be coated or layered with a material to absorb, diffuse, and/or to prevent light from traveling through the lumen 360 of the tubular member 352.

The intraocular implant 350 may also include structural features that aid in anchoring and retaining the implant 350 in the eye. For example, the intraocular implant 350 can include one or more retaining or retention structures, such as protrusions, flanges, collars, wings, etc., that hold and retain the implant in place in the iris of the eye and ensure the proper orientation of the tubular member in the iris. As shown in FIG. 3, the first and second ends of the tubular member may be configured as retaining elements or structures 361 and 362 (e.g. retaining ends). As illustrated, the first and second retaining elements 361 and 362 may each include a circular flange or collar 364 extending around the tubular member 352 near an end. As a result, the tubular member 352 may have the shape substantially similar to a shape of a dumbbell or a bone.

The retaining elements 361 and 362 of the tubular member 352 may be made from shape memory material or hydrogel and may be configured to expand when the tubular member 352 transitions between the first shape and the second shape as described above. The first and second retaining elements 361 and 362 may have a circular cross-section when the tubular member 352 in the second shape or state. In some embodiments, the first and second retaining elements 361 and 362 may have an oval cross-section, a rectangular cross-section, a triangular cross-section, or any other suitable cross-section when the tubular member 352 is in the second shape.

As illustrated, the first and second retaining elements 361 and 362 may be integrally formed with the tubular member 352 as a single piece construction. In some embodiments, the retaining elements 361 and 362 may be manufactured as separate parts and may be assembled onto the tubular member 352. The first and second retaining elements 361 and 362 can be joined to the tubular member 352 by a friction fit or attached with adhesives.

Referring again to FIG. 3, the shaft portion 358 of the tubular member 352 extends between the first and second ends 354 and 356. Although the shaft portion 358 is shown as having a circular cross-sectional shape, the shaft portion 358 may have various cross-sectional shapes (e.g., an oval or rectangular shape) and may vary in cross-sectional shape moving along its length. As illustrated, the shaft portion 358 of the tubular member 352 has a smaller cross-section area than the cross-section of the first and second retaining elements 361 and 362 (e.g., when the tubular member 352 is in the second shape or state). The length of the shaft portion 358 is substantially equal to the thickness of the iris of the eye. As such, the shaft portion 358 of tubular member 352 may be manufactured with different lengths to accommodate the size (e.g., thickness) of the iris of an eye so that the shaft portion 358 has a sufficient length to extend through the iris.

Referring now to FIG. 5, another intraocular implant 550 configured to be implanted in an iris of an eye is illustrated which in many respects corresponds in construction and function to the previously described intraocular implant 350 of FIG. 3. Components of the intraocular implant 550 which generally correspond to those components of the intraocular implant 350 of FIG. 3 are designated by like reference numerals in the five-hundred series. The intraocular implant 550 may reduce the amount or intensity of light that travels through the intraocular implant 550. In some embodiments, the shape of the lumen 560 may absorb light and/or reflect light back toward the anterior chamber. For example, the lumen 560 of the intraocular implant 550 may be rough or uneven to reduce the amount of light that travels through the lumen 560 by absorbing light and/or reflecting light back towards the front of the eye. As illustrated, the surface of the lumen 560 may include a plurality of serrations or grooves to mitigate light from traveling through the intraocular implant 550. For example, the serrations of the lumen 560 may scatter light and reflect light back toward the anterior chamber of an eye. As a result, the intraocular implant 550 may minimize or mitigate dysphotopsia, such as halos and glare, and other aberrant optical effects.

Referring now to FIG. 6, another intraocular implant 650 configured to be implanted in an iris of an eye is illustrated which in many respects corresponds in construction and function to the previously described intraocular implant 350 of FIG. 3. Components of the intraocular implant 650 which generally correspond to those components of the intraocular implant 350 of FIG. 3 are designated by like reference numerals in the six-hundred series. The intraocular implant 650 may reduce the amount or intensity of light that travels through the intraocular implant 650. For example, the lumen 660 of the intraocular implant 650 may have a curved configuration. As illustrated, the shape of the lumen 660 may have a S-shape configured to mitigate light from traveling through the intraocular implant 650. In other embodiments, the shape of the lumen 660 may be C-shaped, Z-shaped, or any other suitable shape. As a result, the intraocular implant 650 can reduce or mitigate dysphotopsia, such as halos and glare, and other aberrant optical effects.

Referring now to FIG. 7, another intraocular implant 750 configured to be implanted in an iris of an eye is illustrated which in many respects corresponds in construction and function to the previously described intraocular implant 350 of FIG. 3. Components of the intraocular implant 750 which generally correspond to those components of the intraocular implant 350 of FIG. 3 are designated by like reference numerals in the seven-hundred series. As shown in FIG. 7, the intraocular implant 550 may include a plurality of lumens 760. The use of a plurality of lumens in the intraocular implant 550 may limit the amount of light that passes through the intraocular implant 550 as compared to the use of a single lumen having approximately the same cross-sectional area. In some embodiments, the intraocular implant 550 may have up to four (4) lumens, or up to six (6) lumens.

Referring now to FIG. 8, another intraocular implant 850 configured to be implanted in an iris of an eye is illustrated which in many respects corresponds in construction and function to the previously described intraocular implant 350 of FIG. 3. Components of the intraocular implant 850 which generally correspond to those components of the intraocular implant 350 of FIG. 3 are designated by like reference numerals in the eight-hundred series. As shown in FIG. 8, the retaining elements of the intraocular implant 850 may be curved or sloped at a desired angle to reduce the risk of damaging the lens or capsular bag of an eye. In some embodiments, the outer portion or edge of the retaining element may be round to reduce the risk of iris shaving when the retaining element makes contact with the iris, especially at the posterior iris that can result in pigment dispersion and clogging of the trabecular meshwork.

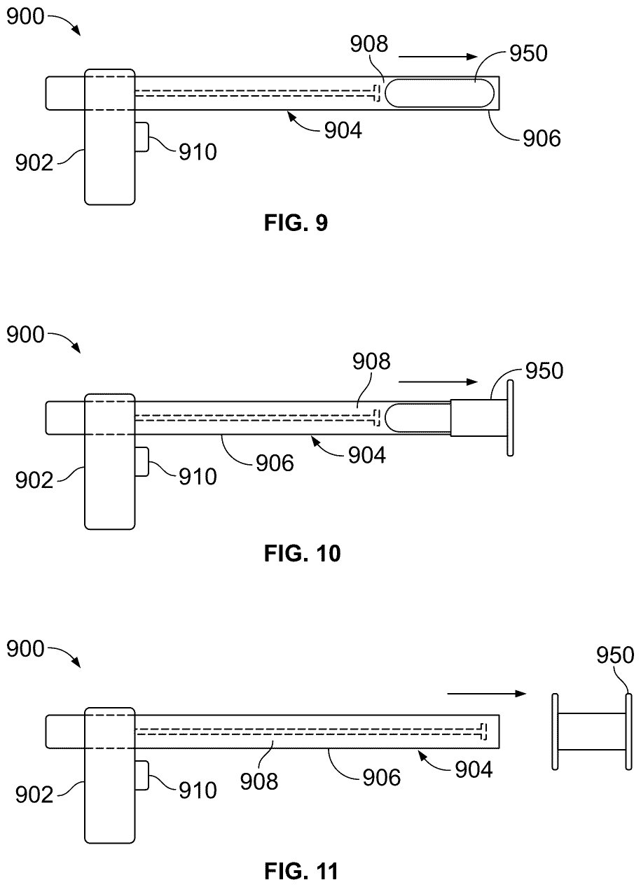

FIGS. 9-11 show a partial view of an exemplary surgical instrument 900 that can be used to implant an intraocular implant 950 into an eye of a patient. Prior to inserting the intraocular implant 950 into the eye, a small slit or opening is made in a portion of the iris of the eye. The incision may be made in the cornea near the limbus. The surgical instrument 900 may have a sharp point at the tip or distal end that may be used to create the opening in the iris. In some embodiments, the intraocular implant 950 may be formed with a sharp point to puncture and create the opening in the iris. Once the opening is made in the iris, a surgeon may use the surgical instrument 900 (e.g., a delivery or injection device) to implant the intraocular implant 950 into an opening in the iris. The surgical instrument 900 may include a handle component 902 and a delivery component 904. Although a straight or linear delivery component 904 is illustrated, it will be recognized that the delivery component 904 may be curved or bent at a desired angle. By way of example, a curve in the delivery component 904 would have the benefit of entering the iris perpendicular to the surface. The delivery component 904 may include an applicator or elongated member (e.g., sleeve) 906 for delivery of the intraocular implant 950 into the eye. The applicator 906 may include a lumen 908 to hold and maintain the intraocular implant 950 in a compressed or reduced shape (e.g., the first shape or state) during implantation. For example, the intraocular implant 950 may be rolled, folded, and/or compressed to fit within the lumen 908 of the surgical instrument 900 and may be positioned within the lumen 908 as shown in FIG. 9. In some embodiments, the tip or distal end of the application 906 may be tapered or reduced in circumference to facilitate compression of the intraocular implant 950, to reduce the length of the applicator 906, and to facilitate insertion of the intraocular implant 950 in the hole or opening of the iris. The handle component 902 of the surgical instrument 900 may be finger-activated. In some embodiments, the handle component 902 of the surgical instrument 900 may include an actuator 910, such as a button, to control the release of the intraocular implant 950 from the applicator 906 into the iris of the eye. In some embodiments, the surgical instrument 900 may include a twist-type delivery system for implanting the intraocular implant 950 into an opening in the iris of the eye. The twist-type delivery system may provide increased control over the release of the intraocular implant 950.

Once the applicator 906 of the surgical instrument 900 is in position relative to the iris of the eye, the surgeon may press the actuator 910 to cause the intraocular implant 950 to be deployed or ejected from the lumen of the applicator 906 into the opening in the iris. In some embodiments, the actuator 910 may be configured to control the speed of the delivery of the intraocular implant 950 into the opening in the iris. When the intraocular implant 950 is formed from a shape memory material, the applicator 906 may be placed in contact with the anterior side of the iris and the intraocular implant 950 may be pushed through the opening in the iris. In other embodiments, the distal end or tip of the applicator 906 may be positioned through the opening in the iris and retracted while deploying the intraocular implant 950 into the opening of the iris.

FIG. 10 shows the intraocular implant 950 being partially extended from the applicator 906, and partially transitioned from the first shape of a reduced size. Once the intraocular implant 950 is deployed from the applicator 906 as shown in FIG. 11, the intraocular implant 950 can then fully transition from the first shape of a reduced size to the second shape of an expanded size (e.g., increased radial size). For example, the transition in shape of intraocular implant 950 can begin once the implant is deployed from the applicator 906 of the surgical instrument 900 and into an opening in the iris of the eye thereby allowing the material of the intraocular implant to return substantially to its original shape or configuration.

The above description is given for purposes of illustration and explanation. It will be apparent to those skilled in the relevant art that changes and modifications may be made to the invention described above without departing from its scope or spirit. For example, it will be recognized by those skilled in the art that the present invention may be combined with other handpieces. Further, the surgical instrument is not limited to phacoemulsification procedures, but may include any number of other surgical or therapeutic applications.

What is claimed is:

1. An intraocular implant for implantation in an iris of an eye, the intraocular implant comprising:
a tubular member at least partially formed of a shape memory material to enable the tubular member to transition between a first shape and a second shape, the tubular member having a first retaining element, a second retaining element, a shaft portion extending between the first and second retaining elements, and at least one lumen extending through the shaft portion, wherein the shaft portion has a length substantially equal to or less than a thickness of the iris and has a smaller cross-section than the first and second retaining elements when the tubular member is in the second shape.

2. The intraocular implant of claim 1, wherein the first retaining element engages with a first surface of the iris, and wherein the second retaining element engages with a second, opposite surface of the iris to retain the tubular member in an opening though the iris.

3. The intraocular implant of claim 1, wherein the first shape of the tubular member comprises a compressed shape, a reduced shape, a compact shape, a deformed shape, a pre-deployed shape, or a dehydrated shape, and wherein the second shape of the tubular member comprises an uncompressed shape, an expanded shape, an initial shape, an enlarged shape, a deployed shape, or a hydrated shape.

4. The intraocular implant of claim 1, wherein the at least one lumen of the tubular member enables fluid to flow through the tubular member.

5. The intraocular implant of claim 1, wherein the tubular member expands radially outward relative to a longitudinal axis of the tubular member upon implantation of the tubular member in an opening extending through the iris.

6. The intraocular implant of claim 1, wherein the tubular member is configured to change from the first shape to the second shape upon contact with a fluid or upon release from a constraining element.

7. The intraocular implant of claim 1, wherein the tubular member is integrally formed of a one-piece construction.

8. The intraocular implant of claim 1, wherein the shaft portion of the tubular member has a substantial circular cross-section when the tubular member is in the second shape.

9. The intraocular implant of claim 1, wherein the first retaining element is coupled to a first end of the tubular member, and wherein the second retaining element is coupled to a second end of the tubular member.

10. The intraocular implant of claim 1, wherein the tubular member is a self-retaining implant.

11. The intraocular implant of claim 1, wherein the tubular member is configured to transition from the first shape to the second shape upon implantation of in the eye.

12. The intraocular implant of claim 1, wherein the lumen of the tubular member provides a passageway for aqueous humor to flow between an anterior chamber and a posterior chamber of the eye when the tubular member is implanted in the iris.

13. The intraocular implant of claim 1, wherein the tubular member is in the second shape when the first retaining element is expanded, the second retaining element is expanded, and the shaft portion is expanded.

14. The intraocular implant of claim 1, wherein the at least one lumen is configured to reduce light from traveling through the tubular member.

15. The intraocular implant of claim 1, wherein the at least one lumen has an S-shape or curved configuration.

16. The intraocular implant of claim 1, wherein an inner surface of the lumen includes a plurality of serrations or grooves.

17. The intraocular implant of claim 1, wherein the first and second retaining elements are configured to expand upon deployment in the eye.

18. A method of implanting an intraocular implant into an iris of an eye, comprising:
forming an incision in the eye;
inserting a portion of a sleeve of a surgical instrument through the incision in the eye, wherein the sleeve of the surgical instrument is configured to hold and maintain the intraocular implant in a first shape for implantation in the eye, and wherein the intraocular implant is constructed from an expandable or shape memory material;
placing a distal end of the sleeve of the surgical instrument adjacent to or within an opening in the iris; and
deploying the intraocular implant from the sleeve of the surgical instrument into the opening of the iris, wherein the intraocular implant is configured to transition from the first shape to a second shape when intraocular implant is deployed from the sleeve; and
removing the portion of the sleeve of the surgical instrument from the eye.

19. The method of claim 18, further comprising causing the surgical instrument to deploy the intraocular implant into the opening in the iris.

20. The method of claim 18, wherein the first shape of the intraocular implant comprises a compressed shape, a reduced shape, a compact shape, a deformed shape, a pre-deployed shape, or a dehydrated shape, and wherein the second shape of the intraocular implant comprises an uncompressed shape, an expanded shape, an initial shape, an enlarged shape, a deployed shape, or a hydrated shape.
